(19) United States
(12) Patent Application Publication
(10) Pub. No.: US 2022/0175908 A1
(43) Pub. Date: Jun. 9, 2022

(54) Methods for Inducing a Safe Immune Response Against Polio Virus

(71) Applicant: Janssen Vaccines & Prevention B.V., Leiden (NL)

(72) Inventors: Conor Cahill, Brussels (BE); Natasa Papic, Den Haag (NL)

(21) Appl. No.: 17/594,494

(22) PCT Filed: May 14, 2020
(86) PCT No.: PCT/EP2020/063413

§ 371 Date: Oct. 19, 2021

(30) Foreign Application Priority Data
May 16, 2019 (EP) 19175000.9

(51) Int. Cl. A61K 39/13; A61P 31/14

(52) U.S. Cl. CPC A61K 39/13; A61K 2039/5252; A61P 31/14

(57) ABSTRACT

The present invention relates to methods and vaccine compositions for inducing a safe immune response against polio virus in a human subject in need thereof, comprising administering to the subject a composition comprising inactivated Sabin poliovirus (sIPV) strains, wherein the sIPV strains have been produced on PER.C6® cells.

### FIELD OF THE INVENTION

The present invention is in the field of medicine. In particular, this invention relates to methods for inducing a safe immune response against polio virus in human subjects. In addition, the invention relates to vaccine compositions for providing safe induction of effective immunity against poliovirus in human subjects.

### BACKGROUND OF THE INVENTION

Polioviruses are members of the Enterovirus genus of the family Picornaviridae.

Polioviruses are small, non-enveloped viruses with capsids enclosing a single stranded, positive sense RNA genome. There are three types of polioviruses: types 1, 2 and 3. Infections of susceptible individuals by poliovirus can result in paralytic poliomyelitis. Poliomyelitis is highly contagious.

Two different polio vaccines have been developed, the inactivated poliovirus vaccine (IPV) of Salk and the live, attenuated oral poliovirus vaccine (OPV) of Sabin. The broad use of these IPV and OPV vaccines, from the mid 1950's in industrialized countries and in worldwide polio immunization programs in ensuing decades, rapidly decreased the global incidence of poliomyelitis. In 2017, thirty years after WHO's public resolution to eradicate polio, wild poliovirus circulated in only three countries and the number of yearly reported cases of poliomyelitis had fallen from 350,000 to 203. In 2018, as of 6 Nov., 27 cases had been reported in two countries.

The trivalent live OPV vaccine was the cornerstone of that success through its ability to induce herd immunity. Yet, as the end of wild poliovirus transmission seems at hand, the routine use of OPV will also have to come to an end. Indeed, the very same trait that made OPV an efficient protection tool when the disease was highly prevalent, its live replicating nature, carries inherent risks—the rare induction by revertant strains of vaccine associated paralytic poliomyelitis and the potential for circulation of vaccine derived poliovirus—that are antagonistic to the final stages of poliovirus eradication. Consequently, in 2008, the World Health Assembly endorsed the cessation of OPV routine vaccination after polio eradication. Since then, OPV cessation has already taken place for poliovirus type 2. After a last case reported in 1999, poliovirus type 2 eradication was certified in 2015. In the following year, a synchronized switch from trivalent OPV to bivalent (type 1 and 3) OPV was operated in all OPV-using countries, in line with the Polio Eradication and Endgame Strategic Plan of WHO, which in addition foresees that at least one dose of trivalent IPV vaccine be adjoined to the bOPV regimen to maintain immunity against poliovirus type 2.

As eradication draws near, the world needs to prepare for the full withdrawal of OPV from routine immunization and to a generalized use of IPV to protect populations against a possible resurgence of poliovirus. Current levels of IPV vaccine supply are however insufficient for widespread use and the development of new, safer-for-production and affordable IPV vaccines has been called for.

Accordingly, there is a need in the art for improved and affordable methods and vaccines for generating immune responses that are safe, long-acting, and effective against poliovirus type 1, 2 and 3, in human subjects.

### BRIEF SUMMARY OF THE INVENTION

The invention relates to methods for inducing a safe and effective immune response against poliovirus in healthy human subjects.

Accordingly, in one aspect the invention relates to methods for inducing a safe immune response against polio virus in a human subject in need thereof, comprising administering to the subject an effective amount of a composition comprising inactivated Sabin poliovirus (sIPV) strains, wherein the sIPV strains have been produced on PER.C6® cells.

In certain embodiments, the immune response comprises the induction of neutralizing antibodies against the wild-type Salk strains (Type 1 [Mahoney], Type 2 [MEF-1] and/or Type 3 [Saukett]), as well as against the Sabin strains (Types 1, 2 and/or 3).

In another aspect, the invention relates to vaccine compositions comprising inactivated Sabin poliovirus (sIPV) strains, wherein the sIPV strains have been produced on PER.C6® cells for inducing a safe immune response against polio virus in a human subject in need thereof.

### BRIEF DESCRIPTION OF THE DRAWINGS

FIG. 1: Participants disposition in the trial.

FIG. 2: Local (A) and systemic (B) solicited adverse events reported during the 7 days after vaccination.

FIG. 3: Geometric Mean Titers (and 95% CI) of poliovirus neutralizing antibodies before and after vaccination. Neutralizing titers to Salk (A) or Sabin (B) virus type 1, 2 and 3 are plotted for each group (n=16 per group). Light grey bars show the baseline titers of the subjects before vaccination, dark grey bars show the titers 28 days after vaccination. The Lower Limit of detection of the assay (LLOQ) is 22.7. Seroprotection in the Salk assay is a titer of 8 for each type 1, 2 and 3. Seroprotection titers for Sabin (type 1, 2 and 3) are not defined.

FIG. 4: Reverse cumulative distribution curves of poliovirus neutralizing antibody titers against Salk strains before and 28 days after vaccination. Dotted line=before vaccination; Solid line=28 days after vaccination. The vertical line at 2.7 shows the LLOQ of the assay. Light grey lines=subjects who received sIPV; Dark grey lines=subjects who received cIPV.

### DETAILED DESCRIPTION OF THE INVENTION

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications, and publications cited herein are incorporated by reference as if set forth fully herein.

It must be noted that as used herein and in the appended claims, the singular forms “a,” “an,” and “the” include plural reference unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all.

As used herein, the term “vaccine” refers to a composition containing an active component effective to induce a certain degree of immunity in a subject against a certain pathogen or disease, which will result in at least a decrease, and up to complete absence, of the severity, duration or other manifestation of symptoms associated with infection by the pathogen or the disease.

As progress is being made towards stopping poliovirus transmission, attention has turned to the needs of a polio-free world and available options to reach adequate IPV supply. The use of poliovirus strains with lower biosafety risk has been called for, along with the search for affordability solutions (World Health Assembly 2008).

The present invention provides methods for inducing a safe immune response against polio virus in a human subject in need thereof, comprising administering to the subject an effective amount of a composition comprising inactivated Sabin poliovirus (sIPV) strains, wherein the sIPV strains have been produced on PER.C6® cells.

In the research that led to the invention, it was shown that the vaccine composition was safe and was overall well tolerated. The local and systemic adverse events classically associated with vaccination were reported as mild or moderate in intensity and the only noteworthy observation was their higher frequency in the sIPV group, which may be explained by the high vaccine dose (15:35:112.5 DU).

The reactogenicity and safety data of this study are similar to previous reports obtained with a Sabin-IPV vaccine assessed in adults in Poland (Verdijk et al., Vaccine 31(47): 5531-5536, 2013) and Cuba (Resik et al., Vaccine 32(42): 5399-5404, 2014), although administered at other dosages. As in the current study, adverse effects (AEs) AEs were generally mild and at most moderate in intensity, the main local AE being pain. It should be noted however that although it is currently not possible to compare antigenic contents of Sabin-IPV vaccines, the establishment of the first WHO international standard will make this feasible in the future (WHO, Expert Committee on Biological Standardization Geneva, 2018).

The robust boosting of polio neutralizing antibody levels observed in this study was also reported for adults immunized in infancy and childhood in Poland and Cuba (Verdijk et al., Resik et al., supra). Although direct comparison is not possible due to probably different total antigenic contents of the vaccines, the post-vaccination GMTs in the Polish adult study and in the current study were very similar. The antibodies elicited by sIPV vaccination neutralized both the wild poliovirus and the Sabin strains.

According to the invention, the poliovirus strains have been produced on PER.C6® cells, as described in WO2011/006823, disclosing the very efficient propagation of poliovirus in PER.C6® cells, wherein unprecedented high titers of poliovirus are obtained. The obtaining of such high titers provides a significant economic advantage over production of poliovirus in Vero cells, which are the conventionally used cells. According to the present invention it has now for the first time been demonstrated that the poliovirus strains produced on PER.C6® cells in high quantities can be used as vaccine compositions and are safe and immunogenic in humans. The IPV vaccine according to the current invention thus offers, in addition to the safety of Sabin strains, an alternative way of increasing production capacity through the high productivity of the PER.C6® cell-culture platform.

PER.C6® cells are immortalized cells, also known in the art as continuous cell lines, and as such have the potential for an infinite lifespan (see e.g. Barrett et al, Expert Rev. Vaccines 8: 607-618, 2009). PER.C6® cells for the purpose of the present application mean the cells as deposited under ECACC no. 9602240 on 29 Feb. 1996. It will be clear to the skilled person that this definition will also include cells from an upstream or downstream passage or a descendent of an upstream or downstream passage of these deposited cells. PER.C6® cells are described in U.S. Pat. No. 5,994,128 and in Fallaux et al., Hum Gene Ther. 9(13):1909-1917 (1998). PER.C6® cells can grow in suspension in the absence of serum, as for instance described in Yallop et al. (Modern Biopharmaceuticals—Design, Development and Optimization. Vol. 3, 2005). It is been demonstrated in WO2011/006823 that these cells are very suitable for production of poliovirus to high levels in serum-free suspension cultures. The use of microcarriers is not required for producing the vaccine compositions used in the current invention, in contrast to the widely used processes with Vero cells. Microcarriers contribute to high costs of poliovirus produced using the conventional Vero cell-based processes.

The polio vaccine compositions used in the present invention comprise inactivated poliovirus strains. They contain the poliovirus D-antigen, which is the important protective antigen. Virus yields can be measured by standard virus titration techniques, while the determination of the D-antigen concentration is also performed by routine techniques well known to the skilled person, e.g. the D-antigen ELISA assay. Potency can be determined using the D-antigen ELISA and by a poliovirus neutralizing cell culture assay on sera from previously immunized rats.

In certain embodiments of the present invention, the vaccine composition comprises inactivated Sabin poliovirus strains of type 1, 2 and/or 3.

In general, each of the poliovirus strains is cultured in a separate process, and if for instance a trivalent vaccine containing all three types of poliovirus is prepared, the inactivated viruses are mixed and formulated for preparation of individual dosages.

In certain embodiments, the vaccine compositions comprise an antigen content of between 2.5 and 20 D-antigen units (DU) of poliovirus Type 1 per human dose. In a particular embodiment, the vaccine composition comprises 15 DU of poliovirus Type 1 per human dose.

In certain embodiments, in the methods of the invention the vaccine composition comprises an antigen content of between 5 and 64 DU of poliovirus Type 2 per human dose. In a particular embodiment, the vaccine composition comprises an antigen content of 35 D-antigen units (DU) of poliovirus Type 2 per human dose.

In certain embodiments, in the methods of the invention the vaccine composition comprises an antigen content of between 12.5 and 112.5 DU of poliovirus Type 3 per human dose. In a particular embodiment, the vaccine composition comprises an antigen content of 112.5 DU of poliovirus Type 3 per human dose.

In certain embodiments for example, a final vaccine per human dose (e.g. 0.5 ml) may for instance comprise 15 D-antigen units (DU) of type 1 poliovirus, 35 DU of type 2 poliovirus and 112.5 DU of type 3 poliovirus, as determined by comparison to reference preparations.

Inactivation of poliovirus can be done according to methods known in the art, for instance with formalin or with β-propiolactone (BPL) (see e.g. Jiang et al., J. Biol. Stand. 14: 103-109, 1986). In certain embodiments, inactivation is performed with formalin, for instance by following the HCHO inactivation process as described in European Pharmacopeia (European Pharmacopoeia 7.0, Poliomyelitis vaccine (inactivated). 04/2010:0214) and WHO (WHO Technical Report Series, No. 910, 2002 Annex 2, Recommendations for the production and control of poliomyelitis vaccine (inactivated)) guidelines.

The purified polio virus is formulated into a pharmaceutical (vaccine) composition. This can be done according to a variety of methods and using a variety of buffers all according to routine methods well known to the person skilled in the art. In general, it entails bringing the polio virus particles in a pharmaceutically acceptable composition, comprising the polio virus and at least a pharmaceutically acceptable excipient. As used herein, the term “pharmaceutically acceptable” means that the carrier or excipient, at the dosages and concentrations employed, will not cause any unwanted or harmful effects in the subjects to which they are administered. Such pharmaceutically acceptable carriers and excipients are well known in the art (see Remington's Pharmaceutical Science (15th ed.), Mack Publishing Company, Easton, Pa., 1980). The preferred formulation of the pharmaceutical composition depends on the intended mode of administration and therapeutic application. The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or non-toxic, non-therapeutic, non-immunogenic stabilizers, and the like. It will be understood that the characteristics of the carrier, excipient or diluent will depend on the route of administration for a particular application. In certain embodiments, the composition may comprise buffered culture medium, which may optionally be Medium M-199, which is used as formulation buffer for certain registered conventional inactivated poliovirus vaccines. Further, phosphate buffered saline may be used, and the final dosage formulations may comprise for instance 0.5% of 2-phenoxyethanol and a maximum of 0.02% of formaldehyde per dose as antimicrobial preservatives.

The polio vaccine used in the methods of the invention can be monovalent, containing one type of poliovirus (type 1, 2 or 3), or divalent (containing two types of poliovirus, e.g. types 1 and 2, 1 and 3 or 2 and 3), or trivalent (containing three types of poliovirus, i.e. types 1, 2 and 3).

The poliovirus vaccines according to the invention can be used as a stand-alone vaccine, but in other embodiments could be combined with other vaccines in the regular manner, e.g. in the form of a combined vaccine against diphtheria, pertussis, tetanus and polio, and can optionally include further vaccine components, e.g. against hepatitis B and/or Haemophilus influenzae, etc.

The poliovirus is suitable for use in the expanded program on immunization (EPI) and can be combined with the vaccines in that program. Similarly to conventional poliovirus vaccines, the vaccine according to the invention can be given as a single dose, or preferably in prime-boost regimens wherein multiple doses of vaccine are administered with appropriate time intervals, e.g. two injections at a 1-2 month time interval, followed by a booster dose 6-12 months later; or for instance an initial oral dose, followed by a second dose about 8 weeks later and optionally a third dose 8-12 months after the second dose; or for instance for infants a first oral dose at 6-12 weeks of age, followed by a second dose about 8 weeks after the first dose and optionally a third dose at about 6-18 months of age; or for example only a single dose for previously vaccinated persons at increased risk; etc.

As used herein, “a method of inducing safe and effective immune response” or “a safe method of inducing an effective immune response” means a method to induce an immune response that is effective to provide benefits of a vaccine, without causing unacceptable vaccine related adverse events, when administered to the human subject.

As used herein, the phrase “unacceptable vaccine related adverse events,” “unacceptable adverse events,” and “unacceptable adverse reaction,” shall all mean harm or undesired outcome associated with or caused by the administration of a vaccine, and the harm or undesired outcome reaches such a severity that a regulatory agency deems the vaccine unacceptable for the proposed use.

As used herein, an “effective immune response” refers to an immune response that is required for or contributes to the prevention of poliovirus infection in a human subject.

As used herein, a “response rate” refers to the number of subjects who have responded to a treatment with a particular outcome divided by the number of treated subjects.

The ability to stimulate a cellular and/or a humoral response can be determined by antibody binding and/or competition in binding (see for example Harlow, 1989, Antibodies, Cold Spring Harbor Press). For example, titers of antibodies produced in response to administration of a composition providing an immunogen, i.e. poliovirus, can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

According to embodiments of the invention, “inducing an immune response” when used with reference to the methods described herein encompasses providing protective immunity and/or vaccinating a subject against an infection, such as a poliovirus infection, for prophylactic purposes.

In an embodiment of the methods of the invention, the induced immune response comprises the induction of neutralizing antibodies against polio viruses. In a preferred embodiment, the induced immune response comprises the induction of mucosal neutralizing antibodies in the nasopharynx and/or intestine. Preferably, the induced antibodies are capable of neutralizing the wild-type Salk strains (Type 1 [Mahoney], Type 2 [MEF-1] and/or Type 3 [Saukett]), in accordance with the World Health Organization (WHO) recommendations for immunogenicity assessment of IPV (Expert Committee on Biological Standardization (ECBS), sixty-fifth report. Recommendations to assure the quality, safety and efficacy of poliomyelitis vaccines (inactivated). WHO Technical Report Series No. 993, Annex 3, 2015).

In certain embodiments herein, at least part of the immune responses induced by the compositions in the regimens disclosed herein are persistent immune responses. An immune response is considered persistent as used herein when the immune response is still significantly above background (e.g., the immune responses measured when placebo is administered instead of the priming and/or boosting compositions as described herein, or the immune response measured just before the first administration of the priming composition) at least 26 weeks, preferably at least 36 weeks, more preferably at least 48 weeks after the last administration of the vaccine compositions. In certain embodiments this can be at least 96 weeks after administration of the first priming composition.

The pharmaceutical compositions can be administered by suitable means for prophylactic and/or therapeutic treatment. Non-limiting embodiments include parenteral administration, such as intradermal, intramuscular, subcutaneous, transcutaneous, or mucosal administration, e.g. intranasal, oral, and the like. In one embodiment, a composition is administered by intramuscular injection. The skilled person knows the various possibilities to administer a pharmaceutical composition in order to induce an immune response to the antigen(s) in the pharmaceutical composition. In certain embodiments, a composition of the invention is administered intramuscularly.

The present invention further relates to vaccine compositions comprising inactivated Sabin poliovirus (sIPV) strains, wherein the sIPV strains have been produced on PER.C6® cells for inducing a safe immune response against polio virus in a human subject in need thereof.

In certain embodiments, the vaccine composition comprises inactivated Sabin poliovirus strains of type 1, 2 and/or 3. The polio vaccine composition can be monovalent, containing one type of poliovirus (type 1, 2 or 3), or divalent (containing two types of poliovirus, e.g. types 1 and 2, 1 and 3 or 2 and 3), or trivalent (containing three types of poliovirus, i.e. types 1, 2 and 3).

In certain embodiments, the composition comprises an antigen content of between 2.5 and 20 D-antigen units (DU) of poliovirus Type 1 per human dose, in particular an antigen content of 15 DU of poliovirus Type 1 per human dose.

In certain embodiments, the composition comprises an antigen content of between 5 and 64 D-antigen units (DU) of poliovirus Type 2 per human dose, in particular an antigen content of 35 D-antigen units (DU) of poliovirus Type 2 per human dose.

In certain embodiments, the composition comprises an antigen content of between 12,5. and 112.5 D-antigen units (DU) of poliovirus Type 3 per human dose, in particular an antigen content of 112.5 D-antigen units (DU) of poliovirus Type 3 per human dose.

The vaccine compositions according to the invention are administered to a human subject, giving rise to an anti-poliovirus immune response in the subject. An amount of a composition sufficient to induce a detectable immune response is defined to be an “immunogenically effective dose.” As shown in the Examples below, the immunogenic compositions of the invention induce a potent humoral immune response.

The vaccine composition can, if desired, be presented in a kit, pack or dispenser, which can contain one or more unit dosage forms containing the active ingredient. The kit, pack, or dispenser can be accompanied by instructions for administration.

The compositions of the invention can be administered alone or in combination with other vaccines, either simultaneously or sequentially depending upon the condition to be treated, and other factors that may affect the treatment.

The invention is further illustrated in the following examples, which are not intended to limit the scope thereof.

### EXAMPLES

### Example 1. Phase 1 Evaluation of a New Sabin IPV Vaccine in Adults

### Study Design

This phase 1, randomized, controlled, double-blind study was conducted at a single study center in Belgium (Center for Vaccinology, Ghent University Hospital). The clinical procedures were approved by the Ethical Committee of the study center and the study was carried out according to good clinical practice principles, in accordance with the declaration of Helsinki. All participants provided written informed consent before undergoing the screening procedures.

The primary objective of the study was to assess the safety and tolerability of sIPV in terms of solicited local and systemic adverse events (AEs) in the 7 days after vaccination, unsolicited events in the 28 days after vaccination and serious adverse events (SAEs) in the 6 months following vaccination. Information on safety laboratory parameters was also collected at screening, immediately before and 7 days after vaccination.

The immunogenicity of the vaccine was assessed as a secondary objective through the measurement of neutralizing antibody titers against the 3 poliovirus types, immediately before and 28 days after vaccination.

### Study Participants:

The study was designed to vaccinate 32 healthy adults between the ages of 18 and 45 years. Participants were randomized into two equal study groups to receive a single dose of the Sabin-IPV vaccine (sIPV) or the conventional Salk-IPV vaccine (cIPV).

Before receiving the study vaccine, participants were screened for eligibility based on physical examination, medical history, vital signs and clinical laboratory tests. The main exclusion criteria from study participation were: polio vaccination in the past 6 months; allergy, hypersensitivity or intolerance to excipients of sIPV or cIPV; immunological deficiency, immune-suppressive treatment or auto-immune disease; any other vaccination received in the 30 days before administration of study vaccine or planned vaccination up to four weeks post-vaccination; acute disease and/or fever at the time of vaccination; investigational drug received within 3 months or an experimental vaccine received within 6 months before administration of study vaccine; participation in another investigational study during the course of this study; pregnant or breast-feeding woman. Women of childbearing potential had to agree to practice a highly effective method of contraception for the study duration.

### Vaccination:

The Sabin-IPV vaccine according to the invention comprised a target antigen content of 15 D-antigen units (DU) for poliovirus Type 1, 35 DU for Type 2, and 112.5 DU for Type 3. The conventional Salk-IPV vaccine was Sanofi Pasteur's Imovax™ polio containing 40 DU of poliovirus Type 1 (Mahoney), 8 DU of Type 2 (MEF-1) and 32 DU of Type 3 (Saukett).

The vaccine was administered in the deltoid muscle. Study vaccines were prepared and administered by designated unblinded study personnel who were not involved in the post-vaccination assessment of participants.

### Safety Analyses:

Safety laboratory assessments were performed on blood samples collected at screening as well as before the administration of the vaccine and 7 days after for the detection of emerging laboratory abnormalities. Laboratory abnormalities were determined according to the FDA/CBER toxicity grading tables (Food and Drug Administration. Center for Biologics Evaluation and Research. Guidance for Industry. Toxicity Grading Scale for Healthy Adult and Adolescent Volunteers enrolled in Preventive Vaccine Clinical Trials, 2007) and the normal ranges of the testing laboratory.

Participants were observed for 30 minutes after vaccination at the study site. Vital signs were measured immediately before and 30 minutes after vaccination. Participants received diary cards for daily recording of their body temperature and occurrences of solicited local and systemic AEs in the 7 days after vaccination. Information on other (unsolicited) AEs and on concomitant medication related to AEs/SAEs was collected at every study visit and contact until Week 4 after vaccination. Information on potential SAEs was collected until 6 months after vaccination

### Immunogenicity Analyses:

A blood sample was collected immediately before and 28 days after vaccination for measuring neutralizing antibody titers against the wild-type Salk strains (Type 1 [Mahoney], Type 2 [MEF-1] and Type 3 [Saukett]), as well as against the Sabin strains (Types 1, 2 and 3) in accordance with the World Health Organization (WHO) recommendations for immunogenicity assessment of IPV (WHO Technical Report Series No. 993, Annex 3, 2015). The assays were performed at the Centers for Disease Control and Prevention (CDC, USA). Polio neutralizing antibodies were determined by 2-fold serial dilution of serum, which was added to HEp-2(C) cells for 5 days, together with Salk Polio virus at 100 CCID50/25 μl, (Mahoney, MEF-1 or Saukett) at 35° C. Cells were stained by Crystal Violet (0.05%) and titer was determined by the mean of the triplicate based on the number of wells positive for neutralization (Weldon et al., Methods Mol. Biol. 1387:145-176, 2016). The assay range was extended to 18.5 log 2 to allow the measurement of unusually high titers induced by boosting pre-immune adults.

### Statistical Analyses:

The primary population for the safety and immunogenicity analyses consisted, respectively, of all vaccinated participants with safety data available and of all vaccinated participants with post-vaccination immunogenicity data.

Regarding demographic characteristics, mean and standard deviation were provided for age, height, weight and body mass index.

Baseline for safety laboratory tests and vital signs was defined as the last evaluation before vaccination. Safety data were analyzed descriptively. The verbatim terms used to identify unsolicited adverse events were coded using the Medical Dictionary for Regulatory Activities (MedDRA version 21). All solicited and unsolicited adverse events with onset within 7 days or 28 days, respectively, after vaccination, including abnormalities in safety laboratory values and vital signs in the 7 days after vaccination, were included in the analysis. For each adverse event, the percentage of participants who experienced at least one occurrence of the event was calculated per vaccine group.

The proportion of participants with antibody titers against poliovirus>8, the seroprotective threshold in the Salk neutralizing assay, and geometric mean titers (GMT) with 95% confidence intervals were calculated per treatment group for each poliovirus type in the two assays. Seroconversion was defined as a post-vaccination antibody titer>8 for initially seronegative participants (titer<8) and a>4-fold increase in antibody titer for those who were initially seropositive (titer>8) (WHO Technical Report Series No. 993, Annex 3, 2015). Reverse cumulative distribution curves of antibody titers were plotted per vaccine group for each poliovirus type.

### Results

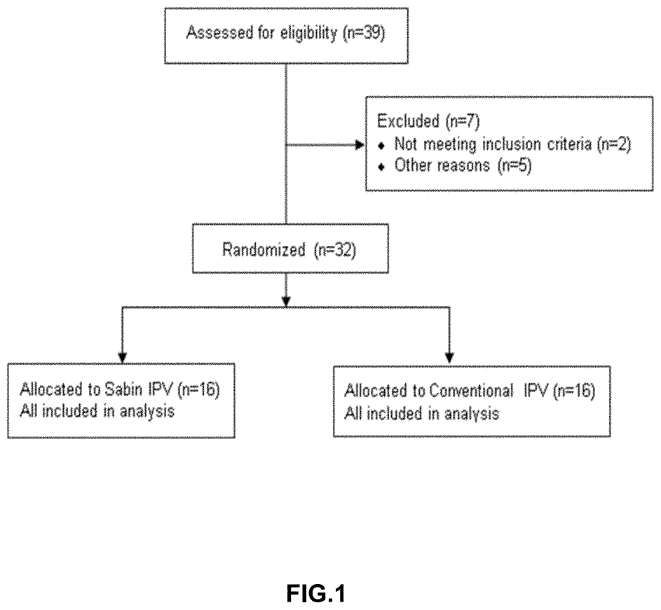

A total of 32 participants were vaccinated and included in the analysis of safety and immunogenicity. The disposition of the participants in the trial is presented in FIG. 1.

### Demographics

Age and other participants characteristics were similar in the two groups (Table 1). More women than men were enrolled in similar proportions in the two study groups.

Polio vaccination of children before the age of 18 months was made mandatory in Belgium in 1967; it is therefore assumed that most enrolled participants had received primary vaccination against poliovirus during childhood. Previous polio vaccination history was not recorded at study entry, but information was sought at the end of the trial from four participants with high pre-vaccination titers and who had not shown a 4-fold increase in titer: two had received an IPV vaccine booster 4 and 5 years before the trial respectively as travel vaccines, the other two participants had no vaccination records nor memory of receiving a polio vaccine booster in preceding years.

TABLE 1
Demographic characteristics of vaccinated participants (mean ± sd)

| | sIPV (N = 16) | cIPV (N = 16) |
|---|---|---|
| Age (year) | 29.8 ± 8.33 | 29.1 ± 8.40 |
| Gender (F/M) | 11/5 | 12/4 |
| Height (cm) | 168.9 ± 8.70 | 169.6 ± 9.56 |
| Weight (kg) | 69.6 ± 9.79 | 67.5 ± 11.18 |
| BMI (kg/m²) | 24.3 ± 3.34 | 23.4 ± 2.63 |

### Safety Results

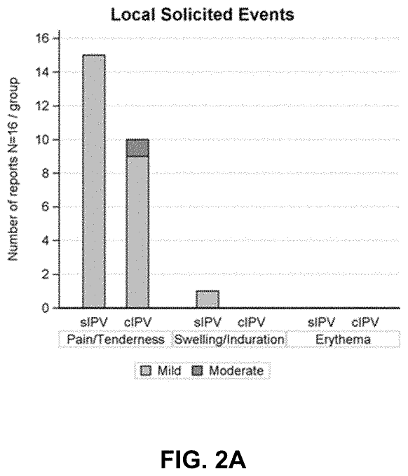

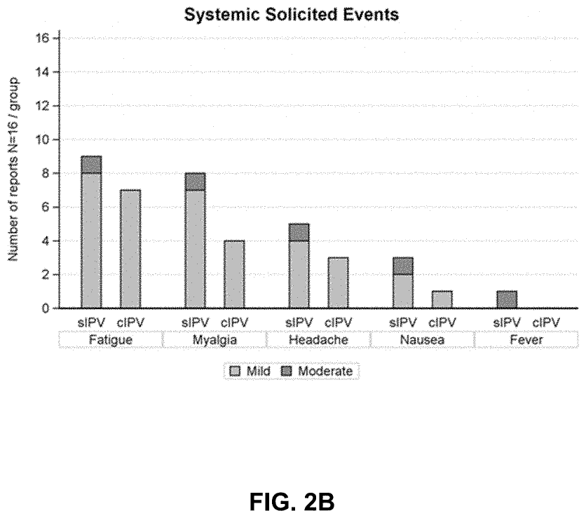

Most study participants, 15 (93.8%) in the sIPV group and 10 (62.5%) in the cIPV group, experienced adverse events (AEs) at the injection site: these were almost exclusively occurrences of pain/tenderness (FIG. 2). A single occurrence of swelling/induration was reported, in the sIPV group, and erythema was not reported. Local solicited AEs were all graded 1 (mild) in intensity, except one occurrence of pain/tenderness in the cIPV group, which was of intensity grade 2 (moderate). A majority of participants in the two groups reported systemic AEs: 13 vaccinees (81.3%) in the sIPV group and 9 (56.3%) in the cIPV group. The most frequent event was fatigue, described by 9 (56.3%) participants in the sIPV group, followed by myalgia, seen in 8 (50%) of the sIPV participants. Systemic AEs judged as caused by vaccination were reported with a frequency of 62.5% in the sIPV group and 50% in the cIPV group. Most solicited systemic AEs were mild in intensity. There was a single occurrence of grade 2 for each of the solicited systemic AEs in the sIPV group. The participant who reported grade 2 fever (38.5° C.-38.9° C.) presented fever on the day of vaccination only; the event lasted one day and was resolved by day 2. It was considered related to vaccination. No grade 3 events were reported during the study. An overall higher incidence of solicited AEs was observed in the sIPV group.

The median time to onset of solicited AEs was similar in the two groups: it was of 1 day for local AEs and 2 days for most systemic events. The median duration was 1 day for all solicited AEs in the two groups.

Other (unsolicited) adverse events were reported by 5 study participants (31.3%) in the sIPV group and by 10 participants (62.5%) in the cIPV group. One occurrence of hemorrhage at the injection site (in the cIPV group) was caused by the vaccination procedure. Unsolicited AEs were mainly represented by respiratory tract infections and headache (11 reports out of 15), and none was judged as causally related to the administration of the study vaccines.

One safety laboratory abnormality emerging after vaccination was detected in each group. This was in both cases a marginal increase in the aspartate aminotransferase (AST) concentration of approximately 1.2-fold compared to the upper limit of normal (ULN), which met the definition of a grade 1 increase (defined as a 1.1-2.5-fold increase).

No SAEs were reported during the 6-months period following vaccination.

### Immunogenicity Results

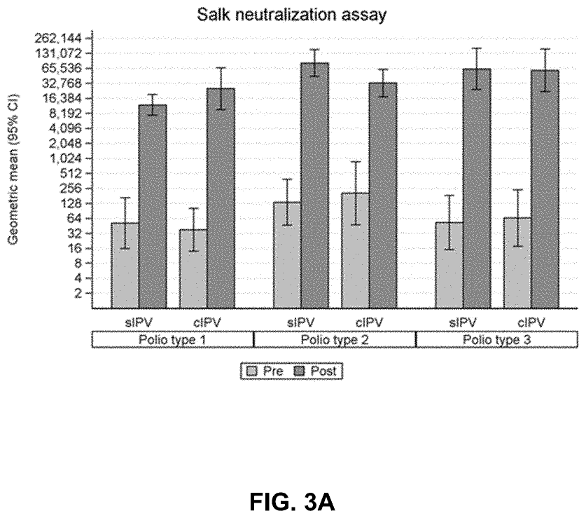

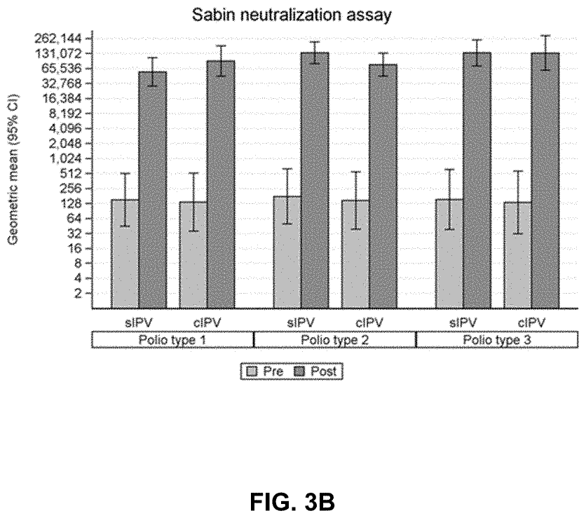

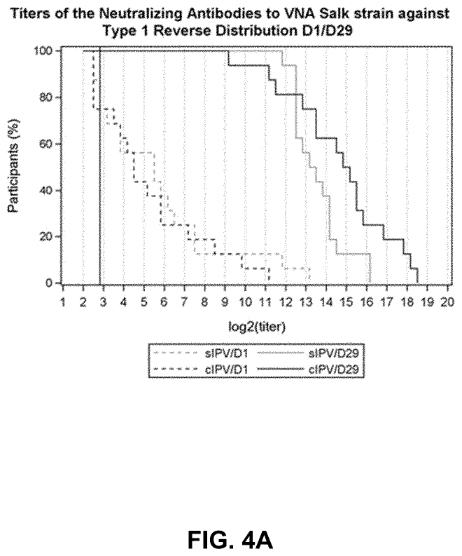

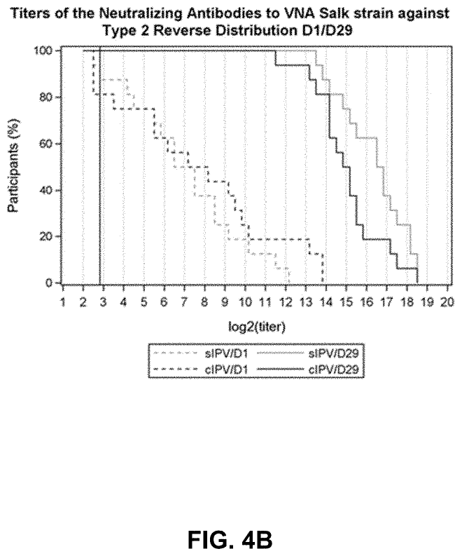

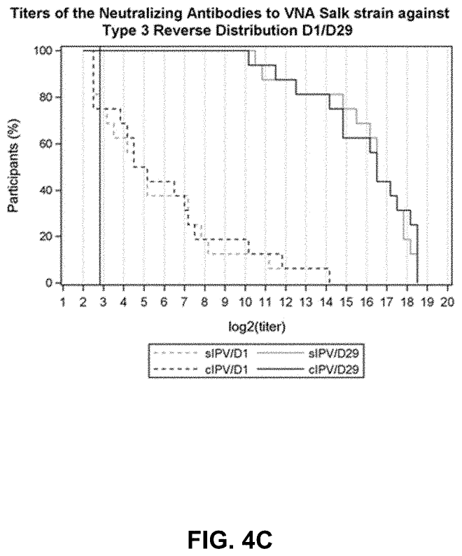

Most participants had seroprotective titers (>8) of neutralizing antibodies against the 3 poliovirus types before vaccination; vaccination with sIPV induced a strong increase in polio neutralizing antibody titers as measured 28 days later (Table 2, FIG. 3). Large titer increases were observed in all subjects except the few who had very high pre-vaccination titers. After vaccination, the few initially seronegative subjects had all mounted antibody titers>8 which defies seroprotection as measured in the Salk assay. The large majority of subjects showed seroconversion, with the exception of those with initially high antibody titers, who did not mount a 4-fold increase in titers.

TABLE 2
Poliovirus neutralizing antibody seroprotection rates and seroconversion rates
for wild type and Sabin poliovirus strains before and 28 days post-vaccination.
Salk Virus Neutralization Assay

| | sIPV | | cIPV | |
|---|---|---|---|---|
| | SP n (%) | SC n (%) | SP n (%) | SC n (%) |
| Type 1 (Mahoney) | | | | |
| Pre (n = 16) | 12 (75.0%) | — | 12 (75.0%) | — |
| Post (n = 16) | 16 (100.0%) | 14 (87.5%) | 16 (100.0%) | 15 (93.8%) |
| Type 2 (MEF-1) | | | | |
| Pre (n = 16) | 14 (87.5%) | — | 13 (81.3%) | — |
| Post (n = 16) | 16 (100.0%) | 15 (93.8%) | 16 (100.0%) | 12 (75.0%) |
| Type 3 (Saukett) | | | | |
| Pre (n = 16) | 12 (75.0%) | — | 12 (75.0%) | — |
| Post (n = 16) | 16 (100.0%) | 16 (100.0%) | 16 (100.0%) | 13 (81.3%) |

Sabin Virus Neutralization Assay

| | sIPV | | cIPV | |
|---|---|---|---|---|
| | SP n (%) | SC n (%) | n ≥ 8 (%) | SC n (%) |
| Type 1 | | | | |
| Pre (n = 16) | 15 (93.8%) | — | 13 (81.3%) | — |
| Post (n = 16) | 16 (100.0%) | 14 (87.5%) | 16 (100.0%) | 15 (93.8%) |
| Type 2 | | | | |
| Pre (n = 16) | 15 (93.8%) | — | 13 (81.3%) | — |
| Post (n = 16) | 16 (100.0%) | 15 (93.8%) | 16 (100.0%) | 15 (93.8%) |
| Type 3 | | | | |
| Pre (n = 16) | 15 (93.8%) | — | 12 (75.0%) | — |
| Post (n = 16) | 16 (100.0%) | 16 (100.0%) | 16 (100.0%) | 15 (93.8%) |

SP (seroprotection = neutralization titer ≥8;
SC (seroconversion) = post-vaccination titer ≥8 in initially seronegative subjects and ≥4-fold increase in titer in initially seropositive subjects

According to the present invention it has been shown that the Sabin-IPV vaccine produced using the high productivity PER.C6® manufacturing platform can make a significant contribution to the availability of safe, effective and affordable poliovirus vaccines for sustained protection of global populations.

1. A method for inducing a safe immune response against polio virus in a human subject in need thereof, comprising administering to the subject a therapeutically effective amount of a composition comprising inactivated Sabin poliovirus (sIPV) strains, wherein the sIPV strains have been produced on PER.C6® cells.

2. The method according to claim 1, wherein the composition comprises inactivated Sabin poliovirus strains of type 1, 2 and/or 3.

3. The method according to claim 1, wherein the composition comprises an antigen content of between 2.5 and 20 D-antigen units (DU) of poliovirus Type 1 per human dose.

4. The method according to claim 3, wherein the composition comprises an antigen content of 15 D-antigen units (DU) of poliovirus Type 1 per human dose.

5. The method according to claim 1, wherein the composition comprises an antigen content of between 5 and 64. D-antigen units (DU) of poliovirus Type 2 per human dose.

6. The method according to claim 5, wherein the composition comprises an antigen content of 35 D-antigen units (DU) of poliovirus Type 2 per human dose.

7. The method according to claim 1, wherein the composition comprises an antigen content of between 12.5 and 112.5 D-antigen units (DU) of poliovirus Type 3 per human dose.

8. The method according to claim 7, wherein the composition comprises an antigen content of 112.5 D-antigen units (DU) of poliovirus Type 3 per human dose.

9. The method according to claim 1, wherein the immune response comprises the induction of neutralizing antibodies against the wild-type poliovirus strains.

10. A vaccine composition comprising inactivated Sabin poliovirus (sIPV) strains, wherein the sIPV strains have been produced on PER.C6® cells, for inducing a safe immune response against polio virus in a human subject in need thereof.

11. The vaccine composition according to claim 10, wherein the composition comprises inactivated Sabin poliovirus strains of type 1, 2 and/or 3.

12. The vaccine composition according to claim 10, wherein the composition comprises an antigen content of between 2.5 and 20 D-antigen units (DU) of poliovirus Type 1 per human dose.

13. The vaccine composition according to claim 12, wherein the composition comprises an antigen content of 15 D-antigen units (DU) for poliovirus Type 1 per human dose.

14. The vaccine composition according to claim 10, wherein the composition comprises an antigen content of between 5 and 64 D-antigen units (DU) of poliovirus Type 2 per human dose.

15. The vaccine composition according to claim 14, wherein the composition comprises an antigen content of 35 D-antigen units (DU) of poliovirus Type 2 per human dose.

16. The vaccine composition according to claim 10, wherein the composition comprises an antigen content of between 12.5 and 112.5 D-antigen units (DU) of poliovirus Type 3 per human dose.

17. The vaccine composition according to claim 16, wherein the composition comprises an antigen content of 112.5 D-antigen units (DU) of poliovirus Type 3 per human dose.
